(19) United States
(12) Patent Application Publication
(10) Pub. No.: US 2003/0229364 A1
(43) Pub. Date: Dec. 11, 2003

(54) Device for anastomosis in a radical retropubic prostatectomy

(76) Inventor: Michael Seiba, El Cajon, CA (US)

Correspondence Address: EASTMAN & ASSOCIATES, 520 West Ash Street, Suite 306, San Diego, CA, 92101, US

(21) Appl. No.: 10/170,022

(22) Filed: Jun. 11, 2002

(51) Int. Cl. A61B 17/08

(52) U.S. Cl. 606/153

(57) ABSTRACT

An anastomosis method and device is disclosed. In one embodiment, an anastomosis device may include a superelastic stent body having a longitudinal cavity that extends from proximal and distal ends of the stent body. Proximal and distal rows of retractable needles having a substantially concave curvature may be circumferentially positioned around the stent body. The device may be configured so that the proximal and distal rows of retractable needles are individually deployable in approximated lumens, such as a urethra and bladder. Once deployed, the proximal and distal rows of retractable needles respectively engage the urethra and bladder.

### FIELD OF THE INVENTION

The present invention relates to a device for joining together two hollow body lumens, and more particularly, to a device for anastomosis in a radical retropubic prostatectomy.

### BACKGROUND OF THE INVENTION

There are many surgical procedures requiring the connection of vessels, hollow organs and other body lumens. While some of these structures are large, and more easily manipulated by the surgeon, other body lumens are smaller and more difficult to manipulate and hold in position while joining ends thereof after, for example, a transectional operation.

Radical retropubic prostatectomy is one type of surgical procedure for patients with localized prostatic carcinoma, and often requires complex and timeconsuming anastomosis. In general, this surgical procedure requires the removal of the prostate gland after severing the gland from the bladder neck and the urethra. It is the attachment of the urethral stump to the bladder neck which is particularly difficult. This difficulty is complicated by the tendency of the urethral stump to retract into adjacent tissue. As a result, considerable time and effort must be extended to re-expose the urethra stump and begin the anastomosis procedure. Further complicating this procedure is the fact that the urethral stump is hidden beneath the pubic bone thus requiring that the surgeon work at a difficult angle and in positions that are uncomfortable and limiting.

While there have been some attempts to provide improved devices and methods for anastomosis in radical retropubic prostatectomy, for example, these attempts have not been entirely successful.

### SUMMARY OF THE INVENTION

In accordance with one aspect of the present invention, an anastomotic device may include a superelastic stent body having a longitudinal cavity that extends from proximal and distal ends of the stent body. Proximal and distal rows of retractable needles having a substantially concave curvature may be circumferentially positioned around the stent body. The device may be configured so that the proximal and distal rows of retractable needles are individually deployable in approximated lumens, such as a urethra and bladder. Once deployed, the proximal and distal rows of retractable needles respectively engage the urethra and bladder.

In accordance with another aspect of the present invention, each of the needles of the proximal and distal rows of retractable needles is positioned at substantially the same distance from adjacent needles.

In another aspect of the present invention, a distance of at least about 1.5 cm separates the proximal and distal rows of retractable needles.

In still yet another aspect of the present invention, the stent body comprises Nitinol alloys.

In another aspect of the present invention, the stent body may be substantially cylindrical.

In yet another aspect of the present invention, the stent body comprises reference markings, half way between the two rows of needles, to facilitate the deployment of the stent body into the urethra and bladder.

In still yet another aspect of the present invention, the proximal and distal rows of retractable needles are permanently affixed to the stent body.

Alternatively, the proximal and distal rows of retractable needles comprise absorbable and/or dissolvable materials.

In yet another aspect of the present invention, the proximal and distal rows of retractable needles each comprise at least four individual needles.

### BRIEF DESCRIPTION OF THE DRAWINGS

The nature, objects, and advantages of the present invention will become more apparent to those skilled in the art after considering the following detailed description in connection with the accompanying drawings, in which like reference numerals designate like parts throughout, and wherein:

FIG. 1 is a side view of a first embodiment of the expandable stent of the present invention;

FIG. 2 is a cross-sectional view of a stent taken along line 2-2 of FIG. 1, showing the spatial relationship of the proximal row of needles relative to the stent body, and one another;

FIG. 3 is a cross-sectional diagram of a typical delivery catheter that may be used to deliver the stent of the present invention to a desired anastomosis site;

FIGS. 4A-E are cross-sectional diagrams showing relevant deployment operations that may be used to deploy the stent of the present invention, providing anastomosis of a urethra and bladder;

FIGS. 5A-C are cross-sectional diagrams showing relevant removal operations that may be used to retrieve the stent of the present invention after anastomosis is completed;

FIG. 6 is a side view of an alternative embodiment of the stent of the present invention;

FIG. 7 is a cross-sectional view of a stent taken along line 7-7 of FIG. 6, showing the spatial relationship of the proximal row of needles relative to the stent body, and one another;

FIG. 8 is a cross-sectional diagram of an alternative delivery catheter that may be used to deliver the stent of the present invention to a desired anastomosis site; and

FIGS. 9A-C are cross-sectional diagrams showing relevant deployment operations that may be used to deploy the stent of the present invention, providing anastomosis of a urethra and bladder.

### DETAILED DESCRIPTION OF A PREFERRED EMBODIMENT

In the following description of a preferred embodiment, reference is made to the accompanying drawings, which form a part hereof, and which show by way of illustration a specific embodiment of the invention. It is to be understood by those of working skill in this technological field that other embodiments may be utilized, and structural as well as procedural changes may be made without departing from the scope of the present invention.

It is to be understood that the stent device and associated methods of the present invention are applicable to a variety of anastomosis procedures wherein two conduits are to be joined in a manner facilitating fluid flow and patency.

Although several stent devices are shown and discussed with reference to the bladder neck and urethral stump as a matter of convenience, it will be appreciated that slight modifications of the device may make the device applicable to other anastomosis procedures, without the need of inventive faculty.

Expandable Stent

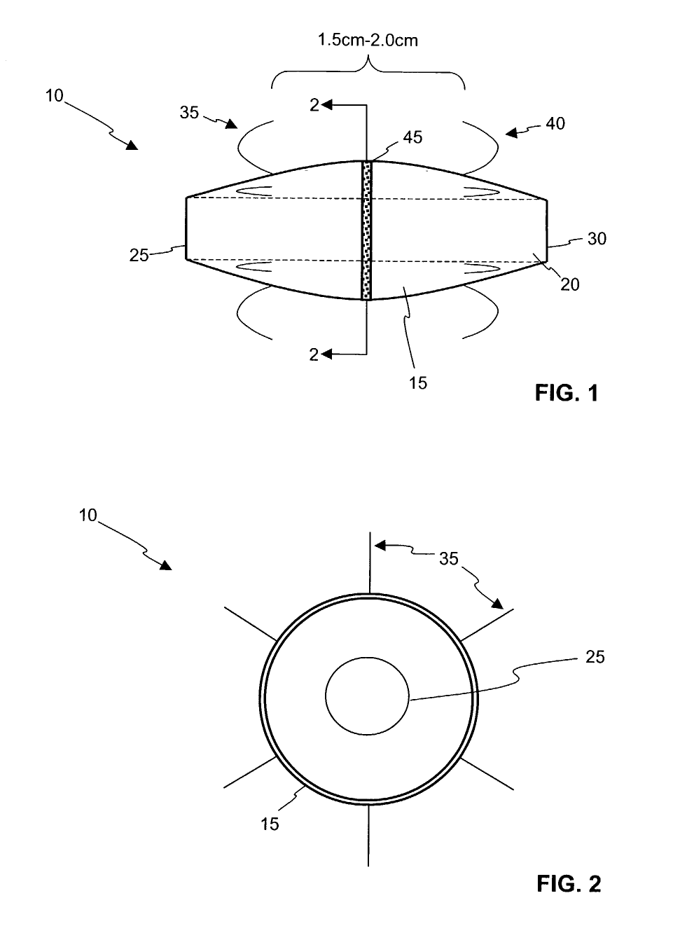

Referring initially to FIG. 1, a side view of a first embodiment of the expandable stent of the present invention is shown and generally designated 10. As shown, stent 10 includes a stent body 15 having a longitudinal cavity 20 that extends from the proximal and distal ends 25, 30 of the stent body 15. Stent 10 may further include two circumferential rows of retractable needles. In particular, stent 10 is shown having proximal and distal rows of needles 35 and 40 that are positioned, respectively, near the proximal and distal ends 25 and 30 of the stent body 15.

The stent body 10 is shown having an optional reference marking 45 that may be positioned, for example, at about the mid-point of the stent body. The reference marking 45 may be implemented as a colored or patterned region that contrasts the surrounding stent body 15. Additionally or alternatively, reference marking 45 may be implemented as ridge, groove, or any other similar spatial identifier. The referenced marking 45 may be used, for example, to facilitate the placement of the stent 10 within a body lumen, which will be described in detail herein.

The row of needles 35 and 40 are typically positioned so there is sufficient distance between these rows to enable the approximation of body lumens. Although the present invention does not rely upon any particular amount of spatial separation between needle rows 35 and 40, an appropriate distance may be anywhere from about 1.5 cm to about 2.0 cm.

Stent 10 may be fabricated using any of a variety of conventional biocompatible materials and processes. Both non-metals and metals can be used. Memory metals are suitable, as well as materials that are absorbable and dissolvable. For example, stent 10 may be fabricated using any of a variety of superelastic or shape memory metals, alloys, plastics, and the like. Currently, Nitinol alloys comprising a mixture of Nickel and Titanium are frequently used in medical device fabrication, and may be used to fabricate the devices of the present invention. Stent 10 may also be fabricated using non-metal materials such as plastics, polyester, polyolefin, nylon, polyurethane, and the like.

If desired, materials that are absorbable by the body once anastomosis is sufficiently completed may be used. Alternatively, dissolvable materials that can pass through the body, for example with different body fluids such as blood, urine, and the like, may also be used.

Stent 10 may be formed using any of a variety of different geometries and configurations including cylindrical, rectangular, oval, and the like. Stent 10 may also be constructed as a wire-like structure (e.g., Nitinol basket), or as a solid or substantially solid design, as long as fluid flow is not unduly hindered.

Each of the individual needles comprising the needle rows 35 and 40 may be fabricated with most any available material, including any of the above-described materials. According to one embodiment, needle rows 35 and 40 may be permanently affixed to the stent body 15, such that they remain attached to the stent body 15 before and after deployment in a body lumen. Alternatively, needle rows 35 and 40 may be made with absorbable and/or dissolvable materials, if 20 desired.

The individual needles of needle rows 35 and 40 are shown with a concave design, with each needle row facing the mid-line of the stent body 15. Typically, the individual needles of rows 35 and 40 are positioned at the same, or substantially the same, distance from one another. This needle arrangement facilitates the placement and retention of stent 10 within a body lumen. However, if desired, a staggered configuration may be used where one or more needles are positioned so that they are closer to, or further away from, the apposing row of needles (not shown).

FIG. 2 is a cross-sectional view of stent 10 taken along line 2-2 of FIG. 1, showing the spatial relationship of the proximal row of needles 35 relative to the stent body 15, and one another. It is to be understood that the distal row of needles 40 may be configured using the same, or different, design used for the proximal row of needles 35.

Stent 10 is shown with six individual needles comprising the proximal row of needles 35. Each of the six needles comprising row 35 are shown positioned at equal distances relative to one another. However, this arrangement is not essential and that individual needles may be arranged so that they are closer to, or further away from, adjacent needles. Although about four to six needles are used in each of the row of needles 35 (and row 40) of a typical stent device, greater or fewer needles may be used.

Delivery Catheter

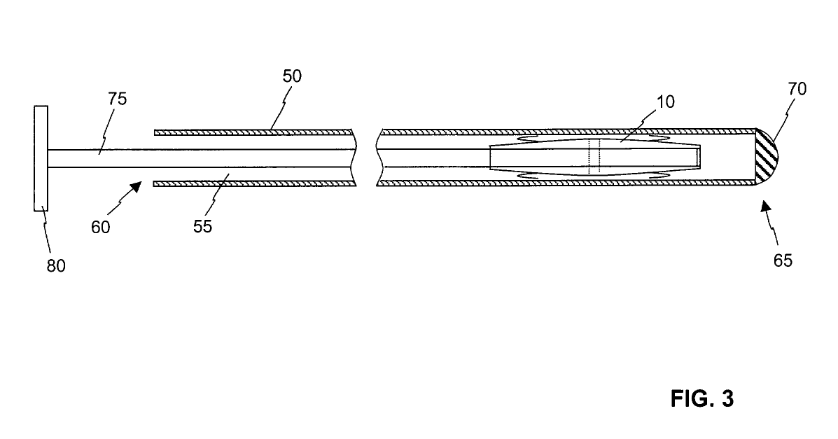

FIG. 3 is a cross-sectional diagram of a typical delivery catheter that may be used to deliver the stent of the present invention to a desired anastomosis site. Delivery catheter 50 represents any of a variety of currently available catheters (e.g., Foley catheter).

Delivery catheter 50 is shown having an elongated tube 55 that has a proximal end 60 that remains outside of a patient's body, and a distal end 65 that is eventually passed through the patient's urethras and into the bladder. The delivery catheter 50 may be configured with an optional removable end cap 70, if desired.

Stent 10 is shown positioned within the catheter tube 55 in a non-deployed state. An appropriate device, such as the push rod 75 and handle 80, may be used to deploy and ultimately recover the stent 10.

Stent Deployment

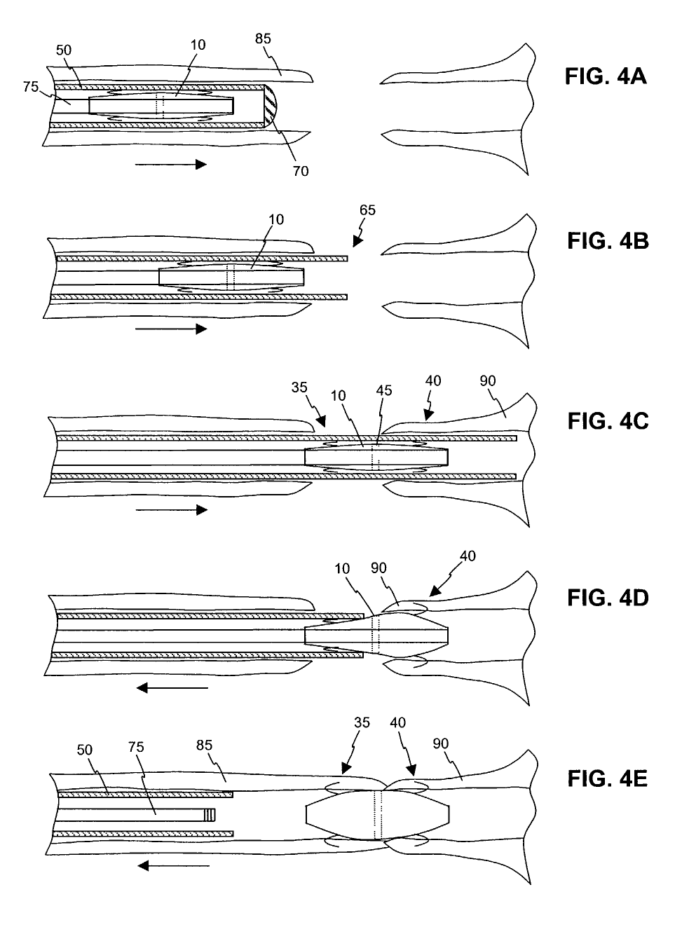

FIGS. 4A-E are cross-sectional diagrams showing relevant deployment operations that may be used to deploy the stent of the present invention, providing anastomosis of a urethra and bladder. For convenience only, the following discussion will reference the anastomosis of a urethra and bladder following a radical retropubic prostatectomy, but it is to be appreciated that the present invention is not so limited and may be utilized in other applications.

To carry stent 10 to the desired anastomosis region, stent 10 may be positioned in its non-deployed state within the delivery catheter 50. As shown in FIG. 4A, the delivery catheter 50 and stent 10 combination may then be advanced through the urethra 85. Advancement of the catheter 50 may continue until distal end 65 of the catheter 50 clears the urethra 85 (FIG. 4B). At this point, the surgeon may remove the optional end cap 70, if necessary.

The catheter 50 may then be further advanced into the bladder 90 (FIG. 4C). Optimally, the positioning of the catheter 50 within the bladder 90 is such that the distal row of needles 40 are contained with the bladder 90, while the proximal row of needles 35 are outside of the bladder 90.

To facilitate the positioning of stent 10, the reference markings 45 on the stent 10 may be used for guidance. For example, reference markings 45 may be located on stent 10 at a position that indicates an optimal or desire depth that stent 10 is to be introduced into the bladder 90. Alternatively, a transparent catheter 50 may permit a surgeon to identify the proximal and distal rows of needles 35 and 40 and then visually estimate a proper insertion depth.

Regardless of the procedure utilized, proper placement of stent 10 within the bladder 90 is critical to successful anastomosis. Once stent 10 has been properly placed within the bladder 90, the delivery catheter 50 may be partially retracted, releasing at least a portion of stent 10, while still containing a remaining portion of stent 10 (FIG. 4D). Specifically, the catheter 50 may be retracted so that the distal rows of needles 40 are deployed, while the proximal rows of needles 35 are still contained with the catheter 50 and remain in an un-deployed state.

Referring still to FIG. 4D, stent 10 is shown partially deployed causing the distal row of needles 40 to be forced in communication with the bladder 90. Typically, the surgeon may manipulate (translate, rotate, etc.) the stent 10 within the bladder 90 to facilitate the proper engagement of the distal row of needles 40 within the bladder 90.

Once acceptable placement of stent 10 within the bladder 90 has been achieved, the urethra 85 and bladder 90 may be brought into approximation (FIG. 4E). At this point, the un-deployed portion of stent 10 (proximal row of needles 35) may be positioned within the urethra 85, while the deployed portion of stent 10 (distal row of needles 40) is positioned within the bladder 90. Next, the delivery catheter 50 may again be retracted so that the remaining (un-deployed) portion of stent 10 can be released.

Similar to the deployment of the distal row of needles 40, the second retraction of the catheter 50 releases the proximal row of needles 35 which are forced into communication with the urethra 85 by the expanding stent 10. Again, it may be necessary for the surgeon to manipulate (translate, rotate, etc.) stent 10 to facilitate the proper engagement of the proximal row of needles 35 within the urethra 85. Rod 75 may then be disengaged from the stent 10 and completely retracted along with the delivery catheter 50. Accordingly, the present invention provides a method and device for the anastomosis of body lumens without the use of sutures, staples or clamps, and is particularly useful for the anastomosis of the urethra and bladder following prostatectomy.

It is to be further understood that the substantially hollow nature of stent 10 permits the introduction of a variety of different surgical tools at any time during or after deployment. Typical devices may include, for example, cystoscopes, resectoscopes, tubes, Foley catheters, artificial sphincters, and the like.

Stent Removal

After a time period, such as for example, thirty days, anastomosis is essentially complete and stent 10 may be removed. Alternatively, it may have already become absorbed by the body or dissolved and passed through the urine.

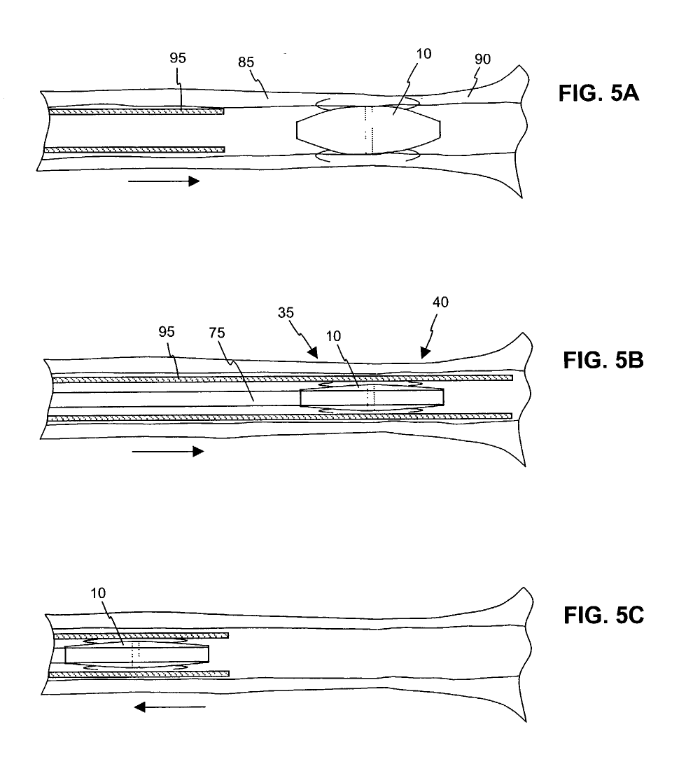

FIGS. 5A-C are cross-sectional diagrams showing relevant removal operations that may be used to retrieve the stent of the present invention. FIG. 5A shows stent 10 in the deployed state, and the advancement of the removal catheter 95 into the urethra 85. The removal catheter 95 may be the same (or different) type of catheter as the delivery catheter. The removal catheter 95 may then be advanced over stent 10, releasing the proximal and distal rows of needles 35 and 40 from their respective positions within the urethra 85 and bladder 90. An appropriate device, such as rod 75, may then engage stent 10 so that the stent and removal catheter 95 may be completely retracted (FIG. 5C).

Although the invention may be implemented using the exemplary stent deployment and removal techniques shown in FIGS. 4A-E, and 5A-C, those of ordinary skill in the art will realize no particular stent deployment and retrieval technique or device is required.

Cylindrical Stent Design

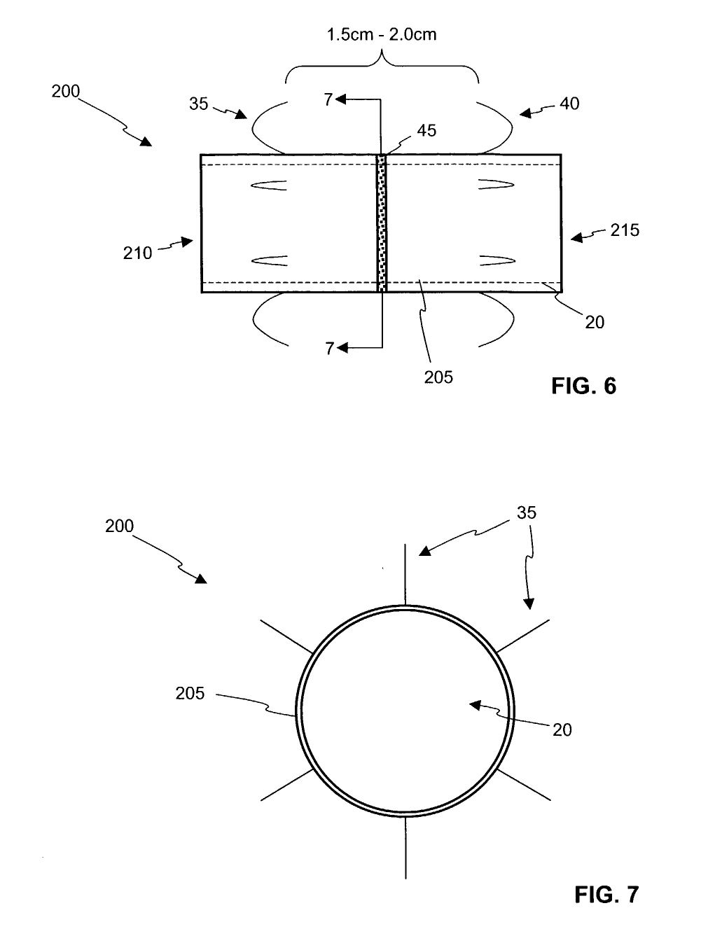

Referring now to FIG. 6, a side view of an alternative embodiment of the expandable stent of the present invention is shown and generally designated 200. Similarly to the stent shown in FIG. 1, stent 200 shown in FIG. 6 comprises a stent body 205 having a longitudinal cavity 20 that extends from the proximal and distal ends 210, 215 of the stent body 205. Stent 200 may also further include proximal and distal rows of needles 35 and 40. Stent body 205 may also include an optional reference marking 45, as previously described. However, in contrast to other stent designs, stent 200 comprises a cylindrical, or substantially cylindrical, structure. Stent 200 and may be constructed using any of the previously described stent construction materials, such as superelastic and shape memory metals, alloys, plastics, and the like.

FIG. 7 is a cross-sectional view of stent 200 taken along line 7-7 of FIG. 6, showing the spatial relationship of the proximal row of needles 35 relative to the stent body 205, and one another. Again, it is to be understood that the distal row of needles 40 may be configured using the same or different design used for the proximal row of needles 35. Stent 200 may also include any of the needle configurations that can be utilized in the other stent designs, as previously described.

Alternative Delivery Catheter

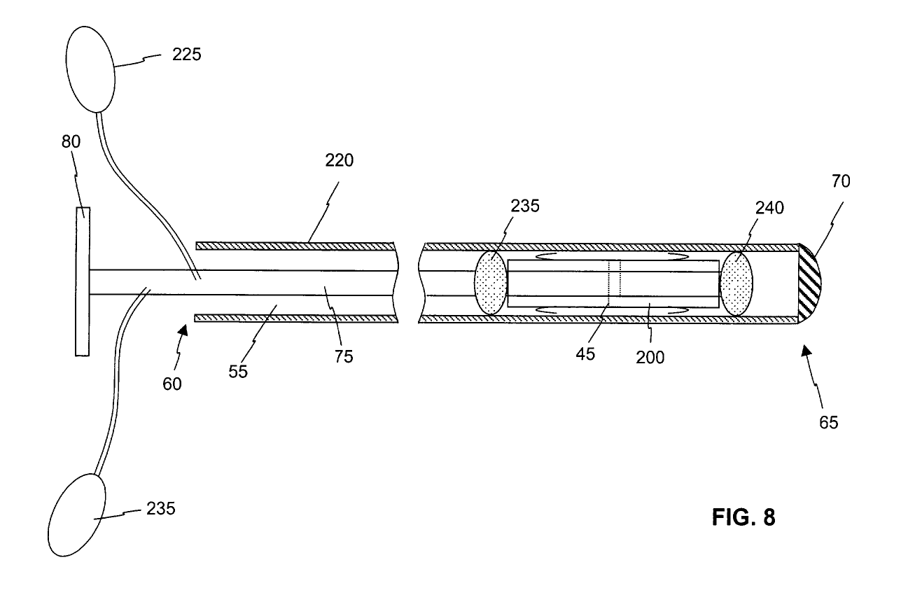

FIG. 8 is a cross-sectional diagram of an alternative delivery catheter that may be used to deliver the stent of the present invention to a desired anastomosis site. Delivery catheter 220 is similar in many respects to the catheter shown in FIG. 3.

However, a notable distinction between these catheters is that the push rod 75 is shown configured with balloon inflation devices 225, 230 which may be used to respectively inflate/deflate balloons 235, 240 using, for example, an appropriate liquid or gaseous medium. One purpose of the balloons 235 and 240 is to facilitate the deployment and recovery of stent 200.

Alternative Stent Deployment

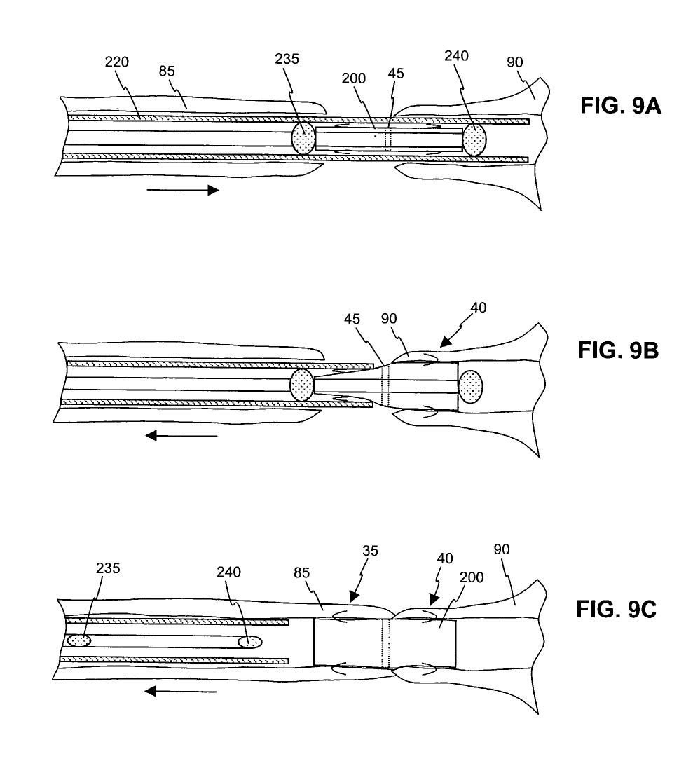

FIGS. 9A-C are cross-sectional diagrams showing relevant deployment operations that may be used to deploy the stent of the present invention, providing anastomosis of a urethra and bladder.

FIG. 9A shows that stent 200 may be positioned in its non-deployed state within the delivery catheter 220 and carried to the desired anastomosis region by advancing these devices through the urethra 85 until the distal end 65 of the catheter 220 is introduced into the bladder 90. Optimally, positioning of the catheter 55 within the bladder 90 is such that the distal row of needles 40 are contained with the bladder 90, while the proximal row of needles 35 are outside of the bladder 90. Once again, reference markings 45 may be used for guidance in positioning the stent.

Once stent 200 has been properly placed within the bladder 90, the delivery catheter 220 may be partially retracted, causing the release of at least a portion of stent 200 while a remaining portion of stent 200 remains contained within the catheter 220. The partial release of stent 200 typically results in the deployment of the distal row of needles 40. If necessary, the surgeon may manipulate (translate, rotate, etc.) the stent 200 within the bladder 90 to facilitate the proper engagement of the distal row of needles 40 within the bladder 90.

Once acceptable placement of stent 200 within the bladder 90 has been achieved, the urethra 85 and bladder 90 may be brought into approximation (FIG. 9C. Next, the delivery catheter 220 may again be retracted so that the remaining (un-deployed) portion of stent 200 can be released, deploying the proximal row of needles 35 which are forced into communication with the urethra 85 by the expanding stent.

Inflatable balloons 235 and 240 may then be deflated using the balloon inflation devices 225, 230. Once deflated, the rod 75 may then be retracted free from the fully deployed stent 200 and completely retracted along with the delivery catheter 220. Removal of stent 200 may be accomplished in a manner similar to that utilized for the other stent embodiments, using, for example, the stent delivery catheter 220.

While there have been shown what are presently considered to be preferred embodiments of the present invention, it will be apparent to those skilled in the art that various changes and modifications can be made herein without departing from the scope and spirit of the invention.

What is claimed is:

1. An anastomosis device comprising:
a superelastic stent body comprising biocompatible material and having a longitudinal cavity that extends from proximal and distal ends of said stent body;
a proximal row of retractable needles having a substantially concave curvature circumferentially positioned around said stent body, wherein said proximal row of said retractable needles is positioned relative to said proximal end of said stent body and facing a mid-line of said stent body;
a distal row of retractable needles having a substantially concave curvature and circumferentially positioned around said stent body, wherein said distal row of said retractable needles is positioned relative to said distal end of said stent body and facing said mid-line of said stent body; and
wherein said proximal and distal rows of retractable needles are individually deployable in approximated first and second body lumens such that once deployed, said proximal and distal rows of retractable of needles respectively engage said first and second body lumens.

2. The anastomosis device according to claim 1, wherein said first body lumen comprises a urethra, and said second body lumen comprises a bladder following a radical retropubic prostatectomy.

3. The anastomosis device according to claim 1, wherein each retractable needle of said proximal and distal rows of retractable needles are positioned at substantially the same distance from adjacent needles.

4. The anastomosis device according to claim 1, wherein a distance of at least about 1.5 cm separates said proximal and distal rows of retractable needles.

5. The anastomosis device according to claim 1, wherein said stent body comprises Nitinol alloys.

6. The anastomosis device according to claim 1, wherein said stent body is substantially cylindrical.

7. The anastomosis device according to claim 1, wherein said stent body is substantially oval.

8. The anastomosis device according to claim 1, wherein said stent body comprises reference markings to facilitate the deployment of said stent body into said first and second body lumens.

9. The anastomosis device according to claim 1, wherein said proximal and distal rows of retractable needles are permanently affixed to said stent body.

10. The anastomosis device according to claim 1, wherein said proximal and distal rows of retractable needles comprise absorbable materials.

11. The anastomosis device according to claim 1, wherein said proximal and distal rows of retractable needles comprise dissolvable materials.

12. The anastomosis device according to claim 1, wherein said proximal and distal rows of retractable needles each comprise at least four individual needles.

13. A method for anastomosis of a urethra to a bladder, said method comprising:
providing an anastomosis device including:
a superelastic stent body comprising biocompatible material;
a proximal row of retractable needles having a substantially concave curvature circumferentially positioned around said stent body and relative to a proximal end of said stent body;
a distal row of retractable needles having a substantially concave curvature and circumferentially positioned around said stent body and relative to a distal end of said stent body;
advancing a catheter containing said anastomosis device through said urethra until a distal end of said catheter clears said urethra and is contained within said bladder;
retracting said catheter to expose a portion of said stent body, wherein said exposed portion of said stent body expands and causes said distal row of retractable needles to engage said bladder; and
retracting said catheter to expose a remaining portion of said stent body, wherein said remaining portion of said stent body expands and causes said proximal row of retractable needles to engage said urethra.

14. The method according to claim 13, said method further comprising:
removing said stent body from said urethra and bladder after said anastomosis is completed.

15. The method according to claim 13, wherein each retractable needle of said proximal and distal rows of retractable needles are positioned at substantially the same distance from adjacent needles.

16. The method according to claim 13, wherein a distance of at least about 1.5 cm separates said proximal and distal rows of retractable needles.

17. The method according to claim 13, wherein said stent body comprises Nitinol alloys.

18. The method according to claim 13, wherein said proximal and distal rows of retractable needles are permanently affixed to said stent body.

19. The method according to claim 13, wherein said proximal and distal rows of retractable needles each comprise at least four individual needles.

20. An anastomosis device comprising:
a stent body comprising biocompatible material and having a longitudinal cavity that extends from proximal and distal ends of said stent body;
a proximal row of retractable needles having a substantially concave curvature circumferentially positioned around said stent body, wherein said proximal row of said retractable needles is positioned relative to said proximal end of said stent body and facing a mid-line of said stent body;
a distal row of retractable needles having a substantially concave curvature and circumferentially positioned around said stent body, wherein said distal row of said retractable needles is positioned relative to said distal end of said stent body and facing said mid-line of said stent body;
a catheter having an elongated tube extending from a proximal end that remains outside of a patient's body, to a distal end that is insertable into a patient's urethra and bladder; and
wherein said proximal and distal rows of retractable needles are individually deployable in said urethra and bladder using said catheter, such that once deployed, said proximal and distal rows of retractable of needles respectively engage said urethra and bladder.
